(19) United States
(12) Patent Application Publication
(10) Pub. No.: US 2025/0166175 A1
(43) Pub. Date: May 22, 2025

(54) METHOD FOR PRODUCING A COSMETIC PRODUCT

(71) Applicant: BASF SE, Ludwigshafen (DE)

(72) Inventors: Christina Kohlmann, Monheim Am Rhein (DE); Lan Liao, Shanghai (CN); Yu hua Ye, Shanghai (CN); Xiao Qing Ing Hu, Shanghai (CN); Jun Tao Xia, Shanghai (CN); Jessica Artioli Centuriao, Monheim Am Rhein (DE); Anja Suckert, Düsseldorf (DE); Christian Huber, Ludwigshafen (DE); Christopher Neary, Monheim Am Rhein (DE); Robert Parker, Ludwigshafen (DE); Zhi Hui Zhu, Shanghai (CN)

(21) Appl. No.: 18/840,125

(22) PCT Filed: Feb. 24, 2023
(86) PCT No.: PCT/CN2023/078229

§ 371 Date: Aug. 21, 2024

(30) Foreign Application Priority Data
Feb. 25, 2022 (WO) PCT/CN2022/077976

(51) Int. Cl. G06T 7/00

(52) U.S. Cl. CPC G06T 7/0012; G06T 2207/30088; G06T 2207/30201

(57) ABSTRACT

Disclosed are methods, apparatuses, systems for generating formulation control data for producing a cosmetic product, wherein an image including a face representation is provided and formulation control data for producing a cosmetic formulation is derived. Further disclosed are cosmetic products and cosmetics formulations produced based on the generated formulation control data.

### TECHNICAL FIELD

Disclosed are methods, apparatuses, systems for generating formulation control data for producing a cosmetic product, wherein an image including a face representation is provided and formulation control data for producing a cosmetic formulation is derived. Further disclosed are cosmetic products and cosmetics formulations produced based on the generated formulation control data.

### TECHNICAL BACKGROUND

Personalized cosmetics is an emerging field including the determination of conditions for cosmetic treatment or the providing of cosmetics according to user's needs. CN111524080A discloses a facial skin feature recognition method to recognize the facial skin of a person and to analyze features to facilitate skin management. KR20220145116A discloses a method for providing a print medium for user-customized color makeup by using an apparatus for providing a print medium for user-customized color makeup. WO2018186666A1 discloses a customized mask pack manufacturing system and manufacturing method based on a 3D model and predetermined functional material.

### SUMMARY OF THE INVENTION

In one aspect disclosed is a method, in particular a computer-implemented method, for generating formulation control data for producing a cosmetic product for treating one or more skin condition(s), the method comprising:
providing at least one image including a face representation,
optionally providing ingredients data associated with formulation components usable to produce the cosmetic product,
detecting at least one facial property associated with one or more sub-area(s) of the face representation,
generating formulation control data by deriving one or more formulation component(s) from the at least one facial property associated with one or more sub-area(s),
providing the formulation control data usable to produce the cosmetic product containing the one or more formulation component(s) preferably per sub-area or sub-area specific.

In another aspect disclosed is an apparatus for generating formulation control data for producing a cosmetic product for treating one or more skin condition(s), the apparatus comprising:
an image provider interface configured to provide at least one image including a face representation,
optionally an ingredients data provider interface configured to provide ingredients data associated with formulation components usable to produce the cosmetic product,
a detector configured to detect at least one facial property associated with one or more sub-area(s) of the face representation,
a generator configured to generate formulation control data by deriving one or more formulation component(s) from the at least one facial property associated with one or more sub-area(s),
a formulation control data interface configured to provide the formulation control data usable to produce the cosmetic product containing the one or more formulation component(s) preferably per sub-area or sub-area specific.

In another aspect disclosed is a method for monitoring one or more skin condition(s), the apparatus comprising:
providing at least one image including a face representation after treatment by the cosmetic product produced based on the formulation control data generated according to the method(s) or apparatus(es) disclosed herein,
providing at least one historical image and at least one facial property detected in one or more sub-area(s) of the corresponding at least one historical image, wherein the at least one historical image was used to generate formulation control data according to the method(s) or apparatus(es) disclosed herein,
detecting at least one facial property associated with one or more sub-area(s) of the face representation,
generating a difference between at least one facial property associated with one or more sub-area(s) of the face representation from the image and the at least one corresponding facial property associated with one or more sub-area(s) of the face representation from the at least one historical image,
providing the generated difference for at least one facial property associated with one or more sub-area(s) of the face representation.

In another aspect disclosed is an apparatus for monitoring one or more skin condition(s), the apparatus comprising:
an image provider interface configured to provide an image including a face representation after treatment with the cosmetic product produced based on the formulation control data generated according to the method(s) or apparatus(es) disclosed herein,
a monitoring data provider interface configured to provide at least one historical image data and at least one facial property detected in one or more sub-area(s) of the corresponding at least one historical image, wherein the at least one historic image was used to generate formulation control data according to the method(s) or apparatus(es) disclosed herein,
a detector configured to detect at least one facial property associated with one or more sub-area(s) of the face representation,
a generator configured to generate a difference between at least one facial property associated with one or more sub-area(s) of the face representation from the image and the at least one corresponding facial property associated with one or more sub-area(s) of the face representation from the at least one historical image,
a difference provider configured to provide the generated difference for at least one facial property associated with one or more sub-area(s) of the face representation.

In another aspect disclosed is a method for producing a cosmetics product, such as a mask or a cosmetic formulation, for treating one or more skin condition(s), the method comprising:
providing formulation control data for producing the cosmetic product as disclosed herein,
producing the cosmetic product according to the formulation control data.

In another aspect disclosed is an apparatus for producing a cosmetics product, such as a mask or a cosmetic formulation, for treating one or more skin condition(s), the apparatus comprising:
a providing interface configured to provide formulation control data for producing the cosmetic product as disclosed herein,
a production apparatus configured to produce the cosmetic product according to the formulation control data.

In another aspect disclosed is a system for producing a cosmetics product, the system comprisesing an apparatus for generating formulation control data as disclosed herein and an apparatus for producing a cosmetics product for treating skin conditions as disclosed herein, wherein the apparatus may be configured to mix formulation components and/or to apply formulation components according to formulation control data.

In another aspect disclosed is the use of the formulation control data to produce a cosmetic product, such as a mask or a cosmetic formulation, for treating one or more skin condition(s).

In another aspect disclosed is a cosmetic product for treating skin conditions produced based on or by using the formulation control data as generated according to the methods or by the apparatuses or systems disclosed herein.

In another aspect disclosed is a computer element, such as a computer readable storage medium, a computer program or a computer program product, comprising instructions, which when executed by a computing node or a computing system, direct the computing node or computing system to provide ingredients data associated with formulation components usable to produce the cosmetic product, wherein the ingredients data is used to generate control data according to the computer-implemented methods disclosed herein.

In another aspect disclosed is a system including:
a computer element, such as a computer readable storage medium, a computer program or a computer program product, comprising instructions, which when executed by a computing node or a computing system, direct the computing node or computing system to provide ingredients data associated with formulation components usable to produce the cosmetic product, wherein the ingredients data is used to generate control data according to the computer-implemented methods disclosed herein, and
one or more capsule(s) each including one or more formulation component(s) usable to produce the cosmetic product based on the ingredients data, wherein the capsule(s) may be configured to be inserted into or placed in one or more apparatus(es) for producing the cosmetic product.

In another aspect disclosed is a computer element, such as a computer readable storage medium, a computer program or a computer program product, comprising instructions, which when executed by a computing node or a computing system, direct the computing node or computing system to carry out the steps of the computer-implemented methods disclosed herein or to provide the formulation control data generated according to the computer-implemented methods disclosed herein.

Any disclosure and embodiments described herein relate to the methods, the apparatuses, the systems, cosmetic products, cosmetic ingredients, uses and the computer elements lined out above and below. Advantageously, the benefits provided by any of the embodiments and examples equally apply to all other embodiments and examples.

### EMBODIMENTS

In the following, embodiments of the present disclosure will be outlined by ways of embodiments and/or example. It is to be understood that the present disclosure is not limited to said embodiments and/or examples.

Determining, generating includes initiating or causing to determine, generate. Providing includes “initiating or causing to access, determine, generate, send or receive”. “Initiating or causing to perform an action” includes any processing signal that triggers a computing node to perform the respective action.

The methods, the systems, apparatuses, cosmetic products, formulation control data and the computer elements disclosed herein enable personalized cosmetics products tailored to the user's need. In particular, the composition of the cosmetic product can be derived from skin diagnostics of the image. This allows to not only treat different sub-areas of the face with different cosmetic products, but to also tailor the cosmetic product itself e.g. by tailoring the active ingredients to be added to a base formulation. For example, the base formulation or the active ingredients making-up the cosmetics product can be adjusted depending on the facial property in the respective sub-area of the face. This way skin can be treated in a more targeted manner. Moreover, the more efficient use of resources potentially combined with the use of natural material results in a positive environmental impact. In particular, the granularity of formulation components for generating control data allows for more tailoring of personalized cosmetics products. In contrast to generating control data for pre-set cosmetic product formulations and only applying such pre-set cosmetic product formulations in a sub-area specific manner, the generation of control data based on formulation components allows for a higher degree of flexibility and scalability with a higher degree of customization.

Formulation component(s) may include any ingredient(s) used to produce a cosmetics product. The formulation component may include a base formulation or an active ingredient. The formulation components may make up the formulation of the cosmetic product. The formulation component may include a base formulation to which one or more active ingredient(s) are to be added according to the generated formulation control data. The base formulation may include one or more formulation ingredients. The formulation component may include one or more active ingredient(s) to be added to the base formulation according to the formulation control data. The formulation components may relate to different sets of active ingredient(s), which may be comprised in base formulation. The formulation component may relate to at least first active ingredient(s) included in base formulation. The formulation component may relate to at least second active ingredient(s) included in base formulation. The formulation components may be compatible to be added together according to the formulation control data. By generating formulation control data for formulation component(s), the flexibility in producing the personalized cosmetic product can be enhanced. Through generation of the formulation control data, the formulation component(s) making up the formulation of the personalized cosmetics product can be adjusted and fine-tuned to the needs of the user.

The base formulation may include one or more formulation ingredients. The base formulation may refer to an ingredient combination suitable to be mixed with one or more active ingredient(s). The base formulation may be a gel such as a water-based based formulation including thickeners, an emulsion such as a water- and oil-based formulation including e.g. a cream or a lotion, a serum such as a water-based, emulsion-based or water-oil based formulation, a cleanser such as a surfactant containing base formulation, micellar water such as a surfactant and oil containing formulation, a hydrogel such as a water-based formulation including thickeners forming a consistent macroscopic structure after drying, an oil, a toner including an effect pigment.

The formulation ingredient, formulation component or active ingredient may be any cosmetically acceptable ingredient. These ingredients are known to the person skilled in the art and can be found in several publications, e.g. in the latest edition of the “International Cosmetic Ingredient Dictionary and Handbook” published by the Personal Care Products Council. Another wellknown source of cosmetically acceptable ingredients is the cometic ingredient database CosIng. CosIng can be accessed via the internet pages of the European Commission.

In one embodiment the at least one base formulation includes at least one ingredient selected from the group comprising or consisting of emulsifier, emollients, waxes, viscosity regulators (thickeners), surfactants, pearlizer, opacifier, sensory enhancers, adjuvants, preservatives, perfumes and combinations thereof.

In one embodiment the at least one base formulation includes at least one ingredient selected from the group comprising or consisting of a stabilizer, a solvent, a solubilizer, a preservative, a neutralizing agent, a buffer, a complexing agent and combinations thereof.

In one embodiment the at least one base formulation includes or is a hydrogel. The hydrogel may include one or more polysaccharide(es). The hydrogel may include Carrageenan gum as Polysaccharide A, and ii) at least one Polysaccharide B selected from the group consisting of Konjac gum, xanthan gum, locust bean gum, Tara gum and guar gum, cellulose gum, microcrystalline cellulose or a mixture thereof. Preferably, the Polysaccharide B consists of one or two polysaccharide gums selected from the group consisting of Konjac gum, locust bean gum and Tara gum; more preferably, the Polysaccharide B consists of Konjac gum. Natural ingredients have little impact on nature as a result of sustainability ecological cultivation.

In one embodiment the at least one active ingredient includes at least one of the following active ingredients: active biogenic ingredients, UV light protection filters, self-tanning agents, insect repellents, antioxidants, film formers, sensory additives, effect pigments, pigments, whitening substances, tyrosine inhibitors (depigmenting agents), coolants, perfume oils, dyes, emollients, surfactants, emulsifiers, humectants, plant extracts, vitamins, peptide and panthenol. The active ingredient may refer to an ingredient suitable to treat the skin condition detectable or detected via the image or associated with the skin condition detectable or detected via the image. The active ingredient allows to treat the skin condition and the methods disclosed herein allow for targeted use of active ingredients. By providing base formulation and active ingredients as formulation components, the cosmetic formulation can be tailored to user's need, preferably sub-area specific.

In one embodiment the facial property relates to or includes at least one skin condition associated with one or more sub-area(s) of the face representation. Detecting the facial property may include generating a score related to the at least one skin condition preferably per sub-area. Detecting the facial property may include generating a score related to a degree of level the at least one skin condition is present preferably per sub-area. Generating formulation control data may include determining one or more formulation component(s) based on the skin condition and/or the score preferably per sub-area. The formulation component(s) may be determined or selected from ingredients data based on the skin condition and/or the respective score preferably per sub-area. For example, if one score of one skin condition is in multiple subareas higher than another score of another skin condition is in other subareas and the formulation components for the two skin conditions are not compatible, the functional component related to the skin condition with higher score may be selected. An amount or a quantity of respective formulation component(s) may be determined based on the score related to the respective skin condition preferably per sub-area. The amount or quantity may be a relative or absolute amount for producing the cosmetic product preferably per sub-area. The amount or quantity may relate to a weight percentage or the relative amount of formulation component per unit area. The score may relate to the degree or level the skin condition is present. For instance, deep forehead wrinkles may be assigned a higher score than fine lip lines or deeper lines may be assigned a higher score than fine lines. The facial property may relate to the representation of the skin detectable via the image. The facial property may be derived from the representation of the face, in particular the representation of the skin. The facial property may relate to or include one or more skin conditions derivable from the representation of the face, in particular the representation of the skin. The skin conditions may be pre-defined. The skin condition may relate to externally detectable skin conditions. The skin condition may relate to skin properties. Skin conditions may include wrinkles, fine lines, pore properties such as pore distribution, black heads or acne, skin appearance such as skin evenness, oily skin or dry skin, oily skin or dry skin, or color properties such as skin type, skin tone, skin tone evenness, redness, spots dark circles or any combinations thereof.

In one embodiment the facial property includes a score related to the at least one skin condition. The score may be derivable or derived from the face representation, in particular the skin representation. The score may be derivable or derived through image processing as described in more detail below. The score may be determined based on image features related to one or more skin condition(s) for one or more sub-area(s). The score may be determined based on image features related to one or more skin condition(s) per sub-area, such as feature level, feature distribution or feature density. For the skin property wrinkles, the score may be determined from image features associated with the geometric properties of the detected winkles. For the skin property pore, the score may be determined from image features associated with the geometric properties and/or color properties of the detected pores, such as distribution of the pores, size of the pores, depth of the pores or color distribution of the pores. For the skin property color, the score may be determined from image features associated with the geometric properties and/or color properties of the detected colors, such as color distribution. For the skin property black heads, the score may be determined from image features associated with the geometric and/or color properties of the detected black heads, such as color distribution, black head area size, black head distribution or blackhead density. For the skin property acne, the score may be determined from image features associated with the geometric properties and/or color properties of the detected acne, such as color distribution, acne area size, acne distribution or acne density. For the skin property redness, the score may be determined from image features associated with the geometric and/or color properties of the detected redness, such as color distribution or redness area size. For the skin property spots, the score may be determined from image features associated with the geometric properties and/or color properties of the detected spots, such as color distribution, spot area size, spot distribution or spot density. For the skin property dark circles, the score may be determined from image features associated with the geometric properties and/or color properties of the detected dark circles, such as degree of darkness, color distribution or area size of dark circles. For the skin property evenness, the score may be determined from image features associated with the geometric properties of the detected evenness. For the skin property oiliness, the score may be determined from image features associated with the geometric and/or color properties of the detected oiliness. For the skin property dryness, the score may be determined from image features associated with the geometric and/or color properties of the detected dryness. Generating a score allows for more tailored assessment of the skin condition. Based on the score the formulation components and/or their respective amount may be determined. By determining the score the cosmetic formulation(s) forming the cosmetic product may be further tailored.

For monitoring one or more skin condition(s), the difference between scores associated with the skin condition preferably per sub-area may be used. The score associated with the skin condition preferably associated with one or more sub-area(s) of the face representation may be determined from at least one current image and at least one historical image. This way a current score and a historical score may be determined preferably per sub-area. The difference of the current and the historical score preferably per sub-area may indicate an evolvement of the skin condition preferably peer sub-area. This way the effectiveness of the treatment may be tracked by a user. Generating formulation control data may include determining a difference between at least one current facial property and at least one historical facial property preferably per sub-area and determining one or more formulation component(s) based on the determined difference between at least one current facial property and at least one historical facial property preferably per sub-area. This way the cosmetics product may be adapted depending on the effectiveness of the treatment. The at least one current facial property may be detected from the face representation of a current image as disclosed herein. The at least one historical facial property may be detected from the face representation of a historical image as disclosed herein. The current image may be the image captured and processed. The historical image may be an image retrieved from a data base storing images of prior facial property detections, preferably including control data generations. Monitoring allows for more targeted treatment over time.

In one embodiment the formulation component relates to a base formulation and/or an active ingredient. At least one base formulation and at least one active ingredient or at least first active ingredient(s) and second active ingredient(s) to be used for producing the cosmetic product may be derived from the at least one facial property associated with one or more sub-area(s). A combination, preferably a sub-area specific combination, of at least one base formulation and one or more active ingredient(s) to be used for producing the cosmetic product or of at least first active ingredient(s) and second active ingredient(s) to be used for producing the cosmetic product may be derived from the at least one facial property, such as the skin condition and/or score, associated with one or more sub-area(s). At least one base formulation and/or at least one active ingredient may be derived from the at least one facial property associated with one or more sub-area(s). More than one active ingredient may be derived for one sub-area, if more than one facial property is detected for one sub-area. Depending on at least one active ingredient derived from the at least one facial property at least one base formulation may be selected for the respective sub-area.

In one embodiment ingredients data associated with formulation components usable to produce the cosmetic product may be provided. The ingredients data may relate the at least one facial property, particularly the skin condition(s) and/or associated scores, to respective formulation component(s). The ingredients data may relate to capsule(s) containing formulation component(s) useable, e.g. to be mixed, applied or simultaneously applied, to produce the cosmetic product. One capsule type may contain one formulation component. One capsule type may contain one base formulation. One capsule type may contain one or more active ingredient(s). One capsule type may contain one or more active ingredient(s) and a base formulation. The ingredients data may relate to different formulation component(s) or respective capsule type(s) or capsule(s) containing formulation component(s) usable to produce the cosmetic product. The ingredients data may relate to different formulation component(s) contained in different capsule(s) or capsule type(s). The ingredients data may relate to laboratory measurement data signifying the compatibility of one or more formulation component(s) e.g. contained in different capsules. This way the ingredients data may signify the compatibility of formulation component(s) or capsule(s). The ingredients data may relate to different formulation component(s) contained in capsules and laboratory measurement data signifying the compatibility of the different formulation component(s) with each other. The ingredients data may include compatibility data e.g. derived from laboratory measurement data and data signifying different formulation component(s) contained in capsules. Compatibility data may signify the compatible formulation component(s). Compatibility may relate to the homogenization behavior, mixing behavior or application behavior of different formulation component(s) contained in different capsules, e.g. when combined. Compatibility may relate to the homogenization behavior, mixing behavior or application behavior of at least one formulation component contained in one capsule and at least one other formulation component contained in another capsule. The formulation control data may be generated based on the ingredients data by selecting one or more formulation component(s), capsule type(s) or capsule(s) based on the at least one facial property, such as the skin condition and/or related scores. The formulation control data may be generated based on the ingredients data by selecting one or more formulation component(s), capsule type(s) or capsule(s) based on their compatibility and/or facial property, such as skin condition and/or score.

In one embodiment deriving formulation control data includes determination of capsules containing different formulation component(s). The formulation control data may be determined from the detected facial property, particularly the skin condition and/or respective scores, The cosmetic product may be produced from the one or more formulation component(s) contained in the capsules. The cosmetic product may be produced from more than one capsule(s), wherein each capsule contains a different formulation component. The cosmetic product may be produced from at least one capsule containing the base formulation and at least one capsule containing at least one active ingredient.

In one embodiment the formulation control data is generated by selecting, based on the at least one facial property associated with one or more sub-area(s), at least one sub-area specific base formulation and/or one or more sub-area specific active ingredient(s) usable to produce a sub-area specific formulation of the cosmetic product. The formulation control data may specify at least one formulation of the cosmetic product per sub-area. The formulation control data may specify one or more formulation components making up the formulation of the cosmetic product, particularly at least one base formulation and at least one active ingredient. The formulation control data may specify a sub-area specific formulation of the cosmetic product with sub-area specific active ingredient(s). The formulation control data may specify at least one quantity, such as an amount of the formulation per sub-area. The formulation control data may specify at least one component of the formulation per sub-area. The formulation control data may specify at least one active ingredient of the formulation per sub-area. The formulation control data may specify at least one component of the cosmetic formulation per sub-area. The formulation control data may specify at least one base formulation of the formulation per sub-area.

The formulation control data may be derived from the skin condition and/or the score for the respective skin condition. The formulation control data, particularly related to the formulation component(s) and/or associated quantities, may be derived from the skin condition and/or the score for the respective skin condition.

In further embodiments the generation of formulation control data may include providing data related to the user and deriving/generating formulation control data based on data related to the user, such as location, time or user specific data. Data related to the user may include weather data or sun exposure data associated with the user's location, pollution exposure data associated with the user's location, the user's age or the user's perceived age as detectable or detected from the image, the user's treatment plan or combinations thereof. Sun exposure or weather data may be used for the generation of formulation control data, e.g. UV filters may be selected as active ingredient. Pollution exposure data may be used for the generation of formulation control data, e.g. polymers shielding the skin, such as natural cationic proteins or antioxidants, may be selected as active ingredient. The user's age may relate to the user's perceived age and may be determined from the image processing by determining the ageing state e.g. from wrinkle score or age spot score derived from the image. The user's age may be used for the generation of formulation control data e.g. active ingredients suitable to treat wrinkles related to the specific age may be selected as active ingredient. The user's treatment plan may be derived from historic images and monitoring of the user's skin condition. The effectiveness of the treatment may be determined from such monitoring data and used for generating formulation control data, e.g. an active ingredient is changed based on the monitoring signifying the ineffectiveness of the prior used active ingredient. The user's treatment plan may be derived from the time the image is captured or the formulation control data is generated and used for generating formulation control data, e.g. at day time UV filters may be selected as active ingredient or at night time oils may be selected as base formulation.

The formulation control data may specify the active ingredient(s) associated with the facial property, such as the skin condition and/or the score related to the respective skin condition.

The formulation data may specify the quantity of active ingredient(s) associated with the facial property, such as the skin condition and the score. The quantity of active ingredient may relate to the weight percentage of active ingredient or the relative amount of active ingredient per unit area. The formulation data, particularly the active ingredient(s) and associated quantities, may be derived from the facial property, such as the skin condition and the score. The active ingredients may be pre-defined. The ingredients data may specify one or more active ingredients per facial property, such as skin condition and/or score. For generation of the formulation control data, one or more active ingredient(s) may be selected from ingredients data based on the facial property, such as skin condition and/or score. The ingredients data may specify one or more base formulation(s) per facial property, such as skin condition and/or score, per sub-area and/or per active ingredient. For generation of the formulation control data, one or more base formulation(s) may be selected from ingredients data based on the facial property, such as skin condition and/or score, the sub-area and/or the active ingredient(s).

In one embodiment the formulation control data specifies at least one base formulation and/or at least one active ingredient associated with one or more sub-area(s). The formulation data, particularly the active ingredient(s), per sub-area may be derived from the facial property, such as the skin condition and the score. The formulation control data, particularly the base formulation, may be derived per sub area e.g. from the facial property, such as the skin condition and/or the score. The base formulation may be pre-defined. The ingredients data may specify more than one base formulation per facial property, such as skin condition and/or score. One base formulation may be selected based on the facial property, such as skin condition and/or score, per sub-area.

The formulation control data may specify the base formulation ingredient(s) associated with the facial property, such as the skin condition and the score. The formulation control data may specify the quantity of ingredient(s) for the base formulation associated with the facial property, such as the skin condition and/or the score. The formulation control data, particularly the ingredient(s) for the base formulation and associated quantities, may be derived from the facial property, such as the skin condition and the score.

In one embodiment multiple facial properties are detected particularly per sub-area, wherein the formulation control data, particularly at least one base formulation and/or one or more active ingredient(s), are derived from the respective facial properties particularly per sub area. For different sub-areas multiple skin conditions may be detected. Scores may be determined for the respective skin condition per sub-area. Based on the skin condition(s) and/or associated score(s) per sub-area formulation control data may be derived. In particular, depending on the skin condition(s) and/or associated score(s) per sub-area active ingredient(s) may be selected for the formulation associated with respective sub-area. This way the formulation can be tailored to the facial properties, such as skin condition(s) and/or associated score(s), detected via the image.

In one embodiment at least one facial property and/or a difference between at least one current facial property and at least one historical facial property is displayed in association with one or more sub-area(s) of the face representation overlaid on the image including the face representation. Multiple facial properties may be detected. For display one or more facial properties may be selected. The selected facial property may be displayed in association with the one or more sub-area(s) of the face representation overlaid on the image including the face representation. For the selected facial property, the difference may be displayed in association with the one or more sub-area(s) of the face representation overlaid on the image including the face representation. The facial property may be displayed by way of skin condition(s) and/or associated score(s). The skin condition to be displayed may be selected by a user. On selection of the skin condition, sub-areas associated with the skin condition may be overlaid on the face representation, where the skin condition is detected. The score(s) or differences based on scores associated with the skin condition may be displayed e.g. by way of scales or bars. The evolvement of the scores or the differences over time may be displayed.

The detecting at least one facial property from the image may include
providing one or more image(s) including a representation of a face,
processing the image by providing at least a portion of the image to a facial recognition model, wherein the facial recognition model is trained to receive at least a portion of the image and, in response to receipt of at least the portion of the image output at least one facial property associated with one or more sub-area(s),
providing at least one facial property associated with one or more sub-area(s).

One or more image(s) may be provided by a camera e.g. of a mobile device, such as front-facing or back-facing camera. The image may be captured via the camera e.g. of a mobile device. The image capture may include providing an image stream of the camera, e.g. the front-facing camera of the mobile device, and an overlaid bounding box signifying the position of the face e.g. to a display of the mobile device. This way the image capture can be guided to capture images required by the recognition model to provide the facial properties. The image may be captured by any device suitable to capture the image. The image may be provided via or from a storage unit to a processing unit configured to detect facial properties.

The facial recognition model may receive at least a portion of the image and, in response to receipt of at least the portion of the image may output one or more facial parameter(s) associated with one or more sub-area(s) of the face representation. The facial recognition model may be a data-driven model trained to receive at least a portion of the image and, in response to receipt of at least the portion of the image to output one or more facial properties or one or more facial parameter(s) mapped to facial properties associated with one or more sub-area(s) of the face representation. The facial recognition model may include a segmentation model for segmenting the face representation and/or one or more sub-area(s) of the face representation. The facial recognition model may include a detection model for detecting at least one facial property associated with one or more sub-area(s) of the face representation.

The image processing may include segmenting at least a portion of the image to extract the face representation and/or one or more sub-area(s) of the face representation. The facial recognition model may include a segmentation model configured to determine a calibration factor preferably based on a reference feature, wherein the calibration factor maps the segmentation of the face representation to the actual size of the face. Detecting of at least one facial property may include image processing by segmenting the image to provide a segmentation of the face representation, a segmentation of the one or more sub-area(s) of the face representation, a segmentation of sub-areas associated with one or more skin condition(s) and/or a calibration factor. The calibration factor may map the segmentation of the face representation, the segmentation of sub-areas associated with one or more skin condition(s) and/or the segmentation of the one or more sub-area(s) of the face representation to the actual size of the face, sub-areas associated with one or more skin condition(s) and/or the one or more sub-area(s) respectively. The segmentation of the image may include processing multiple images to reconstruct the face in three dimensions. This way for instance for a mask the actual face line out can be determined to customize the mask. The segmentation of the image may include processing of a single frontal image. Based on a reference feature the actual size of the face and/or the one or more sub-area(s) may be determined. By using the reference feature the image processing can be reduced to a single image and processing efficiency can be increased or processing power reduced. In addition the calibration factor may be used to tailor the composition, such as amount, or the positioning of the cosmetic formulation. The formulation control data may be generated based on the calibration factor.

The image processing may include determining facial parameters per sub-area and mapping the facial parameters to the at least one facial property. The facial property may relate to a skin condition and/or a score associated with the skin condition. The facial parameters may be determined from image features associated with the skin condition. The score may be determined from the image features associated with the skin condition. The score may be determined from the facial parameters.

In one embodiment the production of the cosmetic product includes mixing the formulation components specified via the formulation control data. For mixing, the formulation control data may specify at least two capsules with different formulation components, such as a first capsule containing the base formulation and at least one second capsule containing active ingredient(s) or such as a first capsule containing first active ingredient(s) and at least one second capsule containing at least second active ingredient(s). The at least two capsules with different formulation components may be provided to a mixing unit. The content of the capsules may be mixed to provide the cosmetic product, such as a cream or a hydrogel containing active ingredients. Multiple formulations may be provided, e.g. multiple hydrogel formulations with different active ingredients to be further processed to a sub-area specific mask, may be provided.

In one embodiment the production of the cosmetic product includes applying, such as sequentially or simultaneously applying, the formulation components according to the formulation control data or applying, such as sequentially or simultaneously applying, mixed formulation components according to the formulation control data. For application, the formulation control data may specify at least two capsules with different formulation components, such as a first capsule containing the base formulation and at least one second capsule containing active ingredient(s) or such as a first capsule containing first active ingredient(s) and at least one second capsule containing at least second active ingredient(s). In one example, the at least two capsules with different formulation components may be provided to an application unit. The base formulation for producing the mask may include a hydrogel. The hydrogel may include different active ingredient(s) per capsule. The formulation component(s) may be the active ingredient(s) contained in the hydrogel. The content of the capsules may be applied, e.g. simultaneously or sequentially, to a base to provide the cosmetic product, such as the mask. Application may include applying different formulation component(s) by applying lines of the different formulation components next to each other or on top of each other preferably specific per sub-area according to the formulation control data. In case of applying lines next to each other, the application may be sequential or simultaneously. This way the exposure of the skin to active ingredients via the mask can be ensured. In case of applying on top of each other, the mixing of active ingredients may occur on application. In another example, the content of the capsules may be mixed. The mixed formulation component(s) may be applied to provide the cosmetic product, such as provided to a base for producing the mask. The base formulation for producing the mask may include a hydrogel. The hydrogel may be mixed with active ingredient(s) according to the formulation data. The mixed hydrogel formulations may be applied preferably specific per sub-area according to the formulation control data.

In one embodiment the cosmetic product is a formulation for at least one sub-area. The formulation control data may include formulation mixing data specifying the formulation for at least one sub-area. A mixing device may include at least one dosing nozzle for releasing at least one base formulation and/or at least one active ingredient. A mixing device may include a mixing unit for mixing at least one base formulation such as an emulsion and at least one active ingredient.

In one embodiment the cosmetic product is a mask including at least one formulation applied to a mask base or including a hydrogel-based formulation applied as mask. The formulation control data may include mask application data specifying the formulation per sub-area. A mask application device may include a mixing unit for mixing at least one base formulation such as a hydrogel and at least one active ingredient. The mask application device may include at least one dosing nozzle for releasing the at least one base formulation and/or at least one active ingredient per sub-area. The mask application device may include a nozzle for applying the at least one base formulation including at least one active ingredient per sub-area.

### BRIEF DESCRIPTION OF THE DRAWINGS

In the following, the present disclosure is further described with reference to the enclosed figures:

FIG. 1 illustrates schematically an example of a mobile device with a front-facing camera for capturing the image of a face.

FIG. 2 illustrates schematically an example of a mobile device with a display for displaying the image to be captured by the front-facing camera and a bounding box to guide the image capture.

FIG. 3 illustrates schematically an example of a flow chart for detecting at least one facial property from an image.

FIG. 4 illustrates schematically an example of a display showing facial properties related to skin conditions and respective scores.

FIG. 5 illustrates schematically an example of a flow chart for generating formulation control data for producing a cosmetic product tailored to sub-areas of the face.

FIG. 6 illustrates schematically an example of an application device for applying a facial mask with cosmetics formulations tailored to sub-areas of the face.

FIG. 7 illustrates schematically an example of a mixing device for mixing a cosmetics formulation to be applied to sub-areas of the face.

FIG. 8 illustrates schematically an example of the methods and apparatuses for producing the cosmetic product.

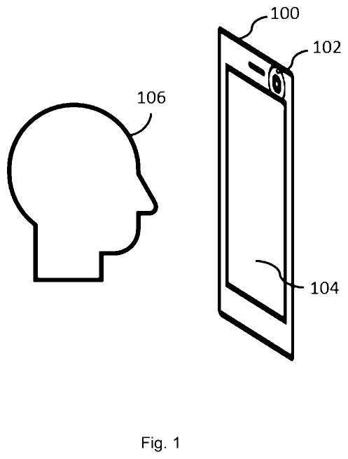

FIG. 1 illustrates schematically an example of a mobile device 100 with a front-facing camera 102 for capturing the image of a face 106 and a display 104 to display the face representation.

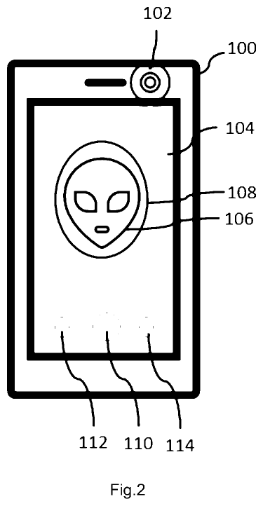

The mobile device 100 includes a display 104 and a camera 102. The camera 102 may be a front-facing camera allowing the user to capture an image of the user's face 106. The image may be provided by a front-facing camera 102 of the mobile device 100. The image may be provided to a display 104. For image capture an image stream may be provided by the front-facing camera 102 to the display 104. This way the image capture of the face may be guided as further illustrated in FIG. 2.

FIG. 2 illustrates schematically an example of a mobile device 100 with a display 104 for displaying the image to be captured by the front-facing camera 102 and a bounding box to guide the image capture.

The image capture may include providing an image stream of the front-facing camera 102 of the mobile device 100. The image stream may be displayed on the display 104. A bounding box 108 may be overlaid on the displayed image stream. The bounding box 110 may signify the position of the face representation on the display 104. This way the user may be guided to capture an image with the representation of the face 106 being placed inside the bounding box 108. For image capture the display 104 may include an interactive touch screen with an image capture bottom 110. Further functionalities for image capture may be provided via further bottoms 112, 114 such as switching to the rear-facing camera or switching from capture mode to the photo library.

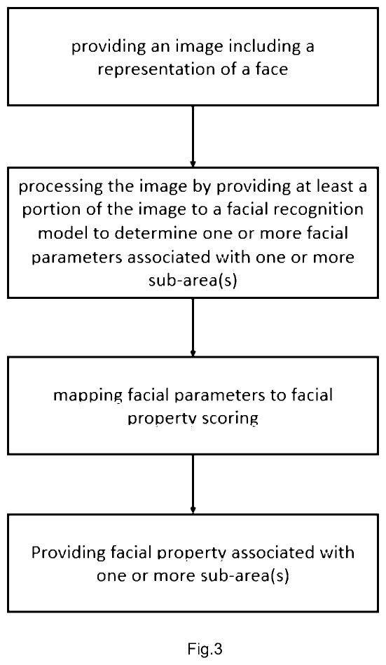

FIG. 3 illustrates schematically an example of a flow chart for detecting at least one facial property from an image.

An image including a representation of a face 106 is provided. The image may be captured via the front-facing or back-facing camera 102 of a mobile device 100 as illustrated in the context of FIGS. 1 and 2. The image capture may include providing an image stream of the front-facing camera 102 and the overlaid bounding box 108 signifying the position of the face representation in the image.

The image may be processed by providing at least a portion of the image to a facial recognition model to determine one or more facial parameter(s) associated with one or more sub-area(s). The facial parameters may be mapped to at least one facial property. The facial property may relate to a skin condition and/or a score associated with the skin condition. The facial parameters may be determined from image features and associated with the skin condition. For instance, wrinkle features may be detected based on facial parameters such as length, depth and width of the wrinkles associated with the face representation or one or more sub-area(s) thereof. Wrinkle features may be further separated by sub-area such as forehead wrinkles, crow's feet, nasobial folds, fine lines between eyebrows or fine lines around eyes. The score per sub-area may be determined from the image features associated with the skin condition. The score may be determined from the facial parameters. For instance, the wrinkle score may be determined based on the detected length, depth and width of the wrinkles.

The facial recognition model may receive at least a portion of the image and, in response to receipt of at least the portion of the image to output one or more facial parameter(s) or facial properties associated with one or more sub-area(s) of the face representation. The facial recognition model may include a segmentation model configured to identify the face representation of the image. The segmentation model may be based on segmentation algorithm. The segmentation model may be configured to segment the face representation and/or sub-areas of the face. The segmentation model may be configured to identify a reference feature such as corneal area of the face representation of the image. The reference feature, such as the size including circumferences, longitudinal axis or horizontal axis of the corneal, may be determined. Based on the reference feature the physical dimensions of the actual face may be calibrated. The corneal area in this embodiment may be used for calibration by mapping the diameter pixel value of the cornea and the standard actual corneal diameter in physical size (11.5 mm). Based on such mapping the pixel size of the other facial areas may be determined and the two-dimensional face representation of only one front face image may be converted to the physical face dimensions (actual or real dimensions face). The segmentation model may provide one or more segments of the face representation including calibration factor for pixel sizes to determine the actual size of the face. One example implementation of such segmentation model is disclosed in CN114863526A. The facial recognition model may further include a detection model configured to detect facial parameters and to map the facial parameters to the facial property including skin condition and/or associated scores.

The facial recognition model may be a data-driven model. The facial recognition model may be trained to receive at least a portion of the image and, in response to receipt of at least the portion of the image to output one or more facial parameter(s) or facial properties associated with one or more sub-area(s) of the face representation. The facial recognition model may be trained based on image data including face representation(s) and associated facial parameter(s). The facial recognition model may be trained based on annotated image data including face representations and facial parameters associated with the respective face representations. The facial parameters may include or be mapped to facial properties such as skin conditions and/or associated scores. The facial recognition model may be based on a neural network structure including at least one encoder and at least one decoder. The facial recognition model may be based on a convolutional neural network structure and/or a recurrent neural network structure. Such neural network structures for image recognition are known in the art and may be trained based on annotated image data.

The facial recognition model may include a segmentation model for segmenting the face representation, one or more sub-area(s) of the face representation and/or one or more sub-area(s) of the face representation associated with skin conditions, and a detection model for detecting at least one facial property associated with one or more sub-area(s) of the face representation. The image processing may include segmenting at least a portion of the image to extract the face representation, one or more sub-area(s) of the face representation associated with skin conditions and/or one or more sub-area(s) of the face representation. The segmentation may include a pixel-based segmentation that classifies pixels into segmentation class(es). The segmentation class may relate to at least one sub-area associated with the face representation and/or skin conditions. The segmentation may include the use of a neural network structure. The segmentation may be based on a neural network structure including at least one encoder and at least one decoder. The segmentation may be based on a convolutional neural network structure and/or a recurrent neural network structure. Such neural network structures for image recognition are known in the art and may be trained based on annotated image data.

The facial parameters may be determined per sub-area and or skin condition of the face representation. The detection model may receive the image or the segmented image. The segmented image may include the segment class and associated pixels. The detection model may be trained to determine facial parameter(s) associated with segment(s) of the image. The detection model may receive the image or the segmented image. The detection model may determine facial parameters. The facial parameters may be mapped to the segments identified according to the segmented image. The facial parameters may be mapped to the respective segment class and associated pixels. The detection model may determine facial parameters and associated sub-areas. The facial parameters and the sub-areas may be mapped to the facial properties, such as skin condition and/or associated score. In other embodiments the facial recognition model may determine the segmentation of the face representation, the facial parameters and associated sub-areas of the face representation.

At least one facial property associated with one or more sub-area(s) may be provided. The facial parameter may be mapped to at least one facial property. The association or mapping between facial parameters and facial properties may be provided by a data base. The facial parameters may relate to facial features. The facial property may relate to the skin condition associated with one or more sub-area(s) of the face representation. The facial property may include a score related to the skin condition. At least one facial property, such as skin condition and/or score, associated with one or more sub-area(s) may be provided. The facial parameter may relate to at least one skin condition and/or score.

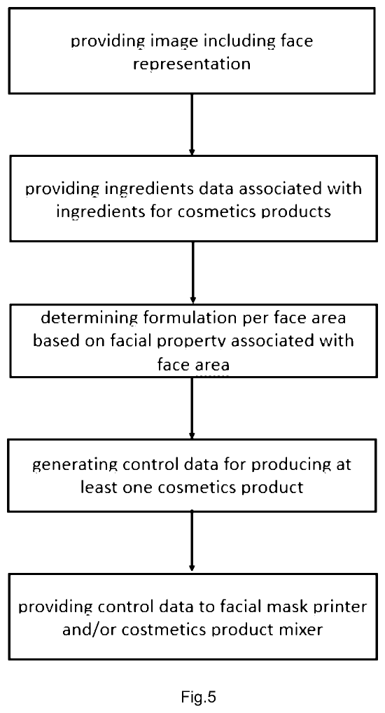

The facial property including the skin condition and/or the score associated with certain subarea(s) of the face representation may be provided. The facial properties determined for different sub-areas may be used to formulate the cosmetic product as will be described in more details in the context of FIGS. 5-8.

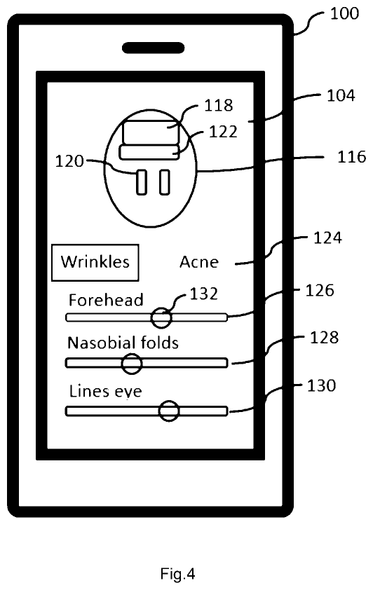

FIG. 4 illustrates schematically an example of a display 104 showing facial properties related to skin conditions and respective scores.

The facial properties 124 may relate to the skin conditions 124 wrinkles and acne. In the example wrinkles are selected. The facial properties 124 may be displayed in association with the sub-areas 118, 120, 122 of the face representation 116. The sub-areas may be forehead 118, nasobial folds 120 and eye lines 122. The sub-areas 118, 120, 122 may be overlaid on the image including the face representation 116. The facial property scores 132 per sub-area and per skin condition may be displayed via bar scales 126, 128, 130 with the scalers 132 signifying the respective scores.

FIG. 5 illustrates schematically an example of a flow chart for generating formulation control data for producing a cosmetic product tailored to sub-areas of the face.

The image including the face representation is provided. Ingredients data associated with formulation ingredients for producing the cosmetic product may be provided. The ingredients data may include a mapping of ingredients and facial properties. The ingredients data may include a mapping of components of the formulation and facial properties. The ingredients data may include a mapping of base formulations to be added to the cosmetic product and facial properties. The ingredients data may include a mapping of active ingredients to be added to the cosmetic product and facial properties.

The ingredients data may be associated with active cosmetic ingredients. Active cosmetic ingredients can be active biogenic ingredients, UV light protection filters, self-tanning agents, insect repellents, antioxidants, film formers, sensory additives, polymers, effect pigments, pigments, whitening substances, tyrosine inhibitors (depigmenting agents), coolants, perfume oils, dyes, emollients, surfactants, emulsifiers, humectants, plant extracts, vitamins, peptide, panthenol or any combination thereof. Active cosmetic ingredients can be active biogenic ingredients, UV light protection filters, self-tanning agents, insect repellents, antioxidants, film formers, sensory additives, effect pigments, tyrosine inhibitors (depigmenting agents), coolants, perfume oils, dyes or humectants. The ingredients data may be associated with emollients. Emollients may be substances that make the skin soft and supple, especially by supplying the skin with lipids or reducing evaporation or increasing the moisture content of the skin. Suitable emollients may be substances from the group of the oils, fats, waxes, hydrocarbons and/or organosilicon compounds that are liquid at room temperature or have a melting point <45° C. Emollients can be oils, fats and/or waxes, for example from the group formed by esters, wax esters, waxes, triglycerides or partial glycerides, natural vegetable oils or fats, hydrocarbons, organosilicon compounds, Guerbet alcohols, mono-/dialkyl ethers, mono-/dialkyl carbonates, and mixtures thereof.

At least one facial property associated with one or more sub-area(s) of the face representation may be determined. The at least one facial property may be detected from the provided image as described in the context of FIGS. 1 to 3. The facial property may be detected in association with the sub-areas of the face.

The at least one facial property, the one or more sub-area(s) of the face representation and the ingredients data may be used to generate formulation control data for producing the cosmetic product. For example, the result of the facial property determination may provide the skin condition, such as wrinkles, the sub-area effected by the respective skin condition, such as wrinkles on the forehead, and the score for the sub-area effected by the respective skin condition. Based on such result the cosmetic ingredients may be selected for producing the cosmetics product.

In the example of wrinkles, the facial property score may reflect the depth of the wrinkles. The active ingredient for producing the cosmetic product may be selected from ingredient data based on the facial property, such as the skin condition and the score. The ingredient data may specify ingredients, ingredients weight percentage and facial properties, such as the skin conditions and the scores. For example, the ingredients data may be associated with active ingredients such as retinol, e.g. retinoic acid, ascorbic acid, hydroxy acids e.g. alpha hydroxy acids (AHAs) such as glycolic, citric and lactic acid, hyaluronic acid, niacinamide such as niacin, Coenzyme Q10, tea extracts, grape seed extracts. The ingredients data may specify for active ingredients the weight percentage to be used in the cosmetics product. The ingredients data may specify compatible combinations of active ingredients. The ingredients data may specify different weight percentages depending on the facial score. For example, if the facial property score indicates fine lines of wrinkles the active ingredient formulation hyaluronic acid may be selected for the respective sub-area.

In the example of acne, the facial property score may reflect the stage of the acne or a skin type. The active ingredient for producing the cosmetic product may be selected from ingredient data based on facial properties. The ingredient data may specify ingredients, ingredients weight percentages for the cosmetic product and facial properties, such as skin conditions and scores. For example, the ingredients data may be associated with active ingredients such as beta-hydroxy acid (BHA), e.g. salicylic acid, antibacterial ingredients, e.g. benzoyl peroxide, sulphur, tree oil, dicarboxylic acid, e.g. azelaic acid, and retinol. The ingredients data may specify for active ingredients the weight percentage to be used for producing the cosmetic product. The ingredients data may specify combinations of active ingredients The ingredients data may specify different weight percentages depending on the scores. For example, if the facial property score indicates an early-stage acne the active ingredient tree-oil and azelaic acid may be selected. If acne and wrinkles are related to the same sub-area, the ingredients data may specify the compatibility. Hence different active ingredients may be selected based on the facial property and their compatibility.

For formulation control data generation, the base formulation such as film forming polymer(s), emollient(s), thickening agent(s), emulsifier for the selected active ingredients may be selected. The base formulation may be selected based on a pre-defined formulation data associated with pre-defined formulations for different active ingredients. The base formulation may be selected based on the facial property and the sub-area. Ingredients data associated with the facial property and pre-defined base formulations for different active ingredients may be used for selection.

For formulation production, the selected active ingredients and/or base formulation may be provided in association with the sub-area of the face. The active ingredients and/or base formulations may be provided in capsules. Per capsule different active ingredients and/or base formulations may be contained. The formulation control data may specify the capsules to be used. For example, different active ingredient combinations may be contained in capsules to be used to produce the cosmetic product.

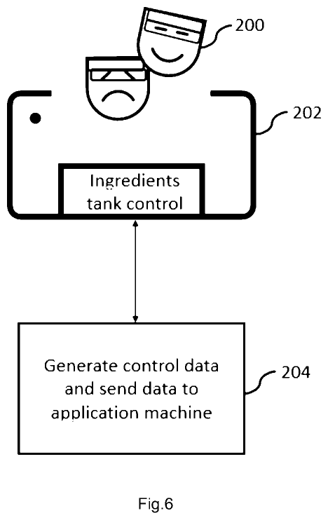

The formulation control data for producing the cosmetic product may be provided. The formulation control data may be provided to an application device for producing facial masks or to a mixing device for mixing the cosmetics formulation as will be described in the context of FIGS. 6 to 8.

FIG. 6 illustrates schematically an example of an application device for applying a facial mask with cosmetic formulations tailored to sub-areas of the face.

The formulation control data may be generated as described in the context of FIGS. 3 and 5. The tailored cosmetic product may be a mask. The mask product may be prepared by using a hydrogel composition. The mask product may include a face and/or body mask, in particular, but not limited to facial mask, neck mask, eye mask, nose mask, hand mask, lip mask and foot mask. The method for producing the mask product may comprise i) preparation of the hydrogel composition in liquid state at a higher temperature, for example 40 to 85° C.; ii) molding the heattreated hydrogel composition (in liquid state) into a target form and obtaining the mask product by cooling to room temperature, thereby allowing gel formation.

Producing the customized mask product may comprise at least one step of applying the hydrogel composition in a nozzle-based application process. The method for preparing the customized mask product may comprise: i) capturing/collecting photographing data of a predetermined area of a user's skin; ii) producing a customized mask pack based on the collected photographing data by using a nozzle-based application machine with the hydrogel composition as ink composition for the machine; iii) cooling the applied hydrogel mask pack at room temperature to achieve gel formation.

According to any one embodiment, the hydrogel composition can be used for preparing the mask product which is customizable for a treatment of all the facial areas. The hydrogel composition can be used for preparing a mask product which is customizable for area-specific treatments.

The formulation control data may include mask application data. The formulation control data may relate to ingredients to be used, the amount or quantity of ingredients to be used, sub-area of ingredients to be used, mask segmentation areas reflecting the segmentation into sub-areas from the face representation. By segmentation of the face representation and reconstruction of the actual face measures, the mask segmentation areas may be customized to the user's face. A formulation control data extract may for instance include the following formulation control data points:

### Mask Segmentation

| Segment sub area | Mask position (pixel) |
|---|---|
| Forehead | 1 to 100 |
| Eyes | 105 to 120 |
| Nasobial folds | 140 to 145 and 155 to 160 |

### Ingredients Control

(percentage per formulation component with respect to mixture of formulation components)

### Forehead Formulation:

Hydrogel base (99%)+Inolixir® (1%) (skin condition: oil control)

### Eye Formulation:

Hydrogel base (99%)+Seanactiv™ (1%) (skin condition: dark circle)

### Nasobial Folds Formulation:

Hydrogel base (98%)+Replexium® (2%) (skin condition: lifting and anti-wrinkle)

Inolixir® is based on chaga mushroom extract as provided by BASF. Seanactiv™ is based on sulfated polysaccharide Fucoidan, an extract of bladderwrack (Fucus vesiculosus) as provided by BASF. Replexium® is based on a blend of two anti-aging tetrapeptidesas as provided by BASF (INCI: Dimethyl Isosorbide (and) Polysorbate 20 (and) Water (and) Acetyl Tetrapeptide-11 (and) Acetyl Tetrapeptide-9).

The formulation control data may be provided to the mask production device 202. The formulation control data may be provided to the ingredients tank control to control release of ingredients during mask production. The ingredients tank may include the capsule containing hydrogel and the capsule for each active ingredient. On applying the hydrogel and the active ingredients may be released sub-area specific based on the formulation control data. Based on the formulation control data a tailored mask with different cosmetic formulations per sub-area may be applied. The mask may be applied to the face of the user reflected in the image representation. This way personalized cosmetics can be even further individualized to the user's needs. Through the tailored cosmetics application the use of ingredient resources can be targeted to the user's needs thus contributing to environmentally friendly cosmetics.

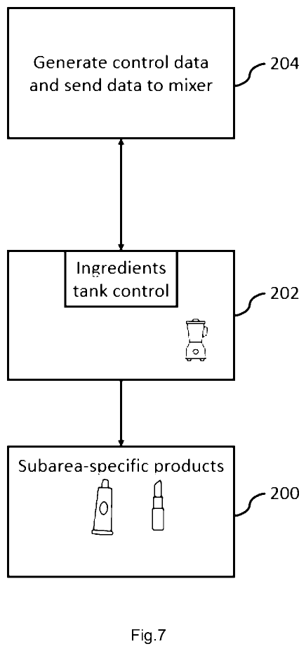

FIG. 7 illustrates schematically an example of a mixing device for mixing a cosmetics formulation to be applied to sub-areas of the face 106.

The formulation control data may be generated as described in the context of FIGS. 3 and 5. The tailored cosmetic product may be the mixed cosmetics formulation. The formulation control data may include formulation mixing data. The formulation control data may relate to ingredients to be used, the amount or quantity of ingredients to be used, sub-area of ingredients target, segmentation areas reflecting the segmentation into sub-areas from the face representation. A formulation control data extract may for instance include the following formulation control data points:

### Ingredients Control

(percentage per formulation component with respect to mixture of formulation components):

### Anti-Spot Serum

Polymer containing water base (99.5%)+DN-Aura® (0.5%) (skin condition: spot, sub-area: cheek)

### Anti-Acne Cream

Emulsion base (99%)+Bix′ Activ® (1%) (skin condition: acne, sub-area: cheek)

### Anti-Black Heads Cleanser

Surfactant containing water base (98%)+BETAPUR® (2%) (skin condition: blackheads, sub-areas: nose and chin)

DN-Aura® is based on extract from the leaves of the Vietnamese tree “Langsat” or “Duku” (Lansium domesticum) as provided by BASF. Bix′ Activ® is based on extract from the seeds of the African Red Lip Tree (Bixa Orellana) as provided by BASF. BETAPUR® is based on extract of “Wild Mint” as provided by BASF.

The formulation control data may be provided to the mixing device 202. The formulation control data may be provided to the ingredients tank control to control release of ingredients for mixing. Based on the formulation control data a cosmetics product 200 with the tailored formulation e.g. for the respective sub-area may be provided. The formulation may be applied to the respective sub-area of the face 106 of the user reflected in the image representation. This way personalized cosmetics can be further individualized to the user's needs. Through the tailored cosmetics application the use of ingredient resources can be targeted to the user's needs thus contributing to environmentally friendly cosmetics.

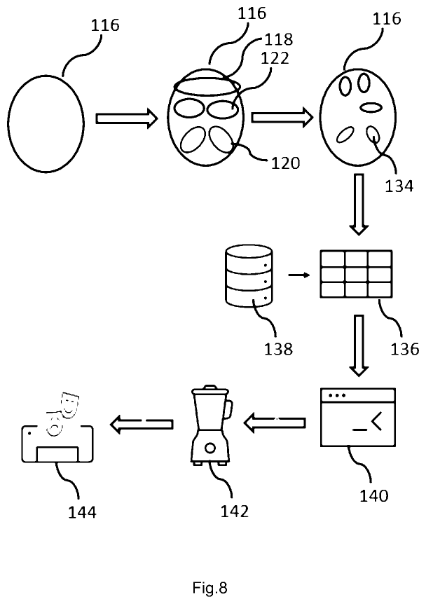

FIG. 8 illustrates schematically an example of the methods and apparatuses for producing the cosmetic product.

The image of the face may be captured. The image may be a front image. The image may be segmented to extract the face representation 116. Further segmentation may include the sub-areas 118, 120, 122. Further segmentation may include the sub-areas 134 related to skin conditions. The segmentation of sub-areas 134 related to skin conditions may be classified according to the skin condition and the sub-area 118, 120, 122 the skin condition is present in. For example, the sub-area may specify the forehead and the skin condition may specify wrinkles and blackheads. Per skin condition and sub-area a score may be determined signifying the strength of the skin conditions. The skin condition and the score per sub-area may form the detected facial property per sub-area.

Ingredient data 136 may be provided by a data base or storage. Ingredient data may be structured according to the formulation components contained in capsules to be mixed and applied for forming the cosmetic product. The ingredient data may specify the skin conditions, score ranges associated with the skin conditions, the sub areas, formulation components contained in capsules to be used for producing the cosmetic product, compatibility measurements for the different formulation components contained in capsules to be used for producing the cosmetic product and quantity measures related to score ranges associated with the skin conditions.

Based on the ingredients data and the detected facial property per sub-area formulations components contained in capsules to be used for producing the cosmetic product may be selected. Upon such selection compatibility measurements may be taken into account. The formulation control data may be generated by a processing unit 140 for the selected formulation components and the respective quantity measures specifying e.g. the relative amount. The formulation data may include mixing data specifying the mixing of formulation components and positioning data signifying the sub-area per mixed formulation component.

Based on the formulation control data the mixing device 142 may select the capsules specified by the formulation control data. The capsules may be identified via a QR code specifying the formulation component(s) included in the capsule. The capsules may be inserted into the mixing device 142. The formulation components contained in the capsules may be mixed. The process of mixing may be executed for more than one formulation component mix. For example, multiple hydrogels with different composition of active ingredients may be mixed per sub area.

The formulation component mix and the formulation control data may be provided to a mask production device 144. The formulation control data may specify the positioning per formulation component mix. For example, hydrogels with different composition of active ingredients may be positioned per sub area. The mask production device 144 may produce the mask based on the formulation control data. The mask may be printed or sprayed. For example, the mask with hydrogels having different composition of active ingredients per sub area may be printed.

The present disclosure has been described in conjunction with preferred embodiments and examples as well. However, other variations can be understood and effected by those persons skilled in the art and practicing the claimed invention, from the studies of the drawings, this disclosure and the claims.

Any steps presented herein can be performed in any order. The methods disclosed herein are not limited to a specific order of these steps. It is also not required that the different steps are performed at a certain place or in a certain computing node of a distributed system, i.e. each of the steps may be performed at different computing nodes using different equipment/data processing.

As used herein “determining” also includes “initiating or causing to determine”, “generating” also includes “initiating and/or causing to generate” and “providing” also includes “initiating or causing to determine, generate, select, send and/or receive”. “Initiating or causing to perform an action” includes any processing signal that triggers a computing node or device to perform the respective action.

In the claims as well as in the description the word “comprising” does not exclude other elements or steps and the indefinite article “a” or “an” does not exclude a plurality. A single element or other unit may fulfill the functions of several entities or items recited in the claims. The mere fact that certain measures are recited in the mutual different dependent claims does not indicate that a combination of these measures cannot be used in an advantageous implementation.

Any disclosure and embodiments described herein relate to the methods, the systems, devices, the computer program element lined out above and vice versa. Advantageously, the benefits provided by any of the embodiments and examples equally apply to all other embodiments and examples and vice versa. All terms and definitions used herein are understood broadly and have their general meaning.

1. A method for generating formulation control data for producing a cosmetic product for treating skin conditions, comprising:
providing an image including a face representation (116),
detecting at least one facial property (124, 132, 134) associated with one or more sub-area (116, 118, 122) of the face representation (116),
generating formulation control data by deriving one or more formulation component from the at least one facial property associated with one or more sub-area (116, 118, 122),
providing the formulation control data usable to produce the cosmetic product (200) containing the one or more formulation component.

2. The method of claim 1, wherein the facial property (124, 132, 134) relates to at least one skin condition (134) associated with one or more sub-area of the face representation (116), wherein detecting the facial property (124, 132, 134) includes generating a score (132) related to the at least one skin condition (134), wherein generating formulation control data includes determining one or more formulation component based on the skin condition (134) and the score (132).

3. The method of claim 1, wherein the formulation component relates to a base formulation and/or an active ingredient, wherein at least one base formulation and at least one active ingredient to be used for producing the cosmetic product (200) or at least first active ingredient and second active ingredient to be used for producing the cosmetic product (200) are derived from the at least one facial property (124, 132, 134) associated with one or more sub-area.

4. The method of claim 1, wherein ingredients data relating to one or more formulation component usable to produce the cosmetic product and/or respective capsule containing formulation component usable to produce the cosmetic product are provided, wherein the ingredients data relate the at least one facial property (124, 132, 134) to respective formulation component and/or respective capsule, wherein the formulation control data is generated based on the ingredients data by selecting one or more formulation component and/or respective capsule based on the at least one facial property (124, 132, 134).

5. The method of claim 4, wherein ingredients data relates to laboratory measurement data signifying the compatibility of one or more formulation component, wherein the formulation control data is generated based on the ingredients data by selecting one or more formulation component based on their compatibility.

6. The method of claim 1, wherein the formulation control data is generated by selecting, based on the at least one facial property (124, 132, 134) associated with one or more sub-area, at least one sub-area specific base formulation and/or one or more sub-area specific active ingredient usable to produce a sub-area specific formulation of the cosmetic product.

7. The method of claim 1, wherein multiple facial properties are detected per sub-area, wherein at least one base formulation and/or one or more active ingredient is derived from the respective facial properties.

8. The method of claim 1, wherein scores are determined for the skin condition per sub-area, wherein sub-area specific formulation control data is derived based on the skin condition and associated score per sub-area.

9. The method of claim 1, wherein generating formulation control data includes determining a difference between at least one current facial property (124, 132, 134) and at least one historical facial property (124, 132, 134) and determining one or more formulation component based on the determined difference.

10. The method of claim 1, wherein detecting at least one facial property (124, 132, 134) includes:
providing an image including a representation of a face,
processing the image by providing at least a portion of the image to a facial recognition model, wherein the facial recognition model is trained to receive at least a portion of the image and, in response to receipt of at least the portion of the image output at least one facial property (124, 132, 134) associated with one or more sub-area, the facial recognition model includes a segmentation model configured to determine a calibration factor based on a reference feature, wherein the calibration factor maps the segmentation of the face representation (116) to the actual size of the face,
providing at least one facial property (124, 132, 134) associated with one or more sub-area, wherein the formulation control data is generated based on the calibration factor.

11. The method of claim 1, wherein at least one facial property (124, 132, 134) and/or at least one difference between at least one current facial property (124, 132, 134) and at least one historical facial property (124, 132, 134) is displayed in association with one or more sub-area of the face representation (116) overlaid on the image including the face representation (116).

12. An apparatus for generating formulation control data for producing a cosmetic product for treating skin conditions, comprising:
an image provider interface configured to provide an image including a face representation (116),
a detector configured to detect at least one facial property (124, 132, 134) associated with one or more sub-area of the face representation (116),
a generator configured to generate formulation control data by deriving one or more formulation component from the at least one facial property (124, 132, 134) associated with one or more sub-area,
a formulation control data interface configured to provide the formulation control data usable to produce the cosmetic product containing the one or more formulation component per sub-area.

13. An apparatus for monitoring a cosmetic condition, comprising:
an image provider interface configured to provide an image including a face representation (116) after treatment with the cosmetic product produced based on the formulation control data generated according to the method of claim 1,
a monitoring data provider interface configured to provide at least one historical image data and at least one facial property (124, 132, 134) detected in one or more sub-area of the corresponding at least one historical image, wherein the historical image was used to generate formulation control data according to the method of claim 1,
a detector configured to detect at least one facial property (124, 132, 134) associated with one or more sub-area of the face representation (116),
a generator configured to generate a difference between at least one facial property (124, 132, 134) associated with one or more sub-area of the face representation (116) from the image and the at least one corresponding facial property (124, 132, 134) associated with one or more sub-area of the face representation (116) from the historical image,
a difference provider configured to provide the generated difference for at least one facial property (124, 132, 134) associated with one or more sub-area of the face representation (116).

14. (canceled)

15. A computer element, which when executed by a computing system, direct computing system to provide ingredients data associated with formulation components usable to produce the cosmetic product, wherein ingredients data is used to generate control data according to the computer-implemented methods of claim 1.
